(19) United States
(12) Patent Application Publication
(10) Pub. No.: US 2025/0074882 A1
(43) Pub. Date: Mar. 6, 2025

(54) IMPROVED PROCESS FOR THE PREPARATION OF 5-[(4-BROMO-2-FLUOROPHENYL)AMINO]-4-FLUORO-N-(2HYDROXYETHOXY)-1-METHYL-1H-BENZIMIDAZOLE-6-CARBOXAMIDE

(71) Applicant: MSN LABORATORIES PRIVATE LIMITED, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (JP); Vijayavitthal T MATHAD, Hyderabad, Telangana (IN); Purna Chandrasekhar Reddy THIPPIREDDY, Hyderabad, Telangana (IN); Sridhar Goud RACHALA, Hyderabad, Telangana (IN); Srimanth Reddy PATLOLLA, Hyderabad, Telangana (IN)

(73) Assignee: MSN LABORATORIES PRIVATE LIMITED, R&D CENTER, Hyderabad, Telangana (IN)

(21) Appl. No.: 18/727,091

(22) PCT Filed: Jan. 6, 2023
(86) PCT No.: PCT/IN2023/050014

§ 371 Date: Jul. 7, 2024

(30) Foreign Application Priority Data
Jan. 6, 2022 (IN) 202241000791

(51) Int. Cl. C07D 235/16; C07F 7/18

(52) U.S. Cl. CPC C07D 235/16; C07F 7/1804

(57) ABSTRACT

The present invention relates to an improved process for the preparation of 5-[(4-bromo-2-fluorophenyl)amino]-4-fluoro-N-(2-hydroxyethoxy)-1-methyl-1H-benzimidazole-6-carboxamide represented by the following structural formula-1, which is referred to as Binimetinib

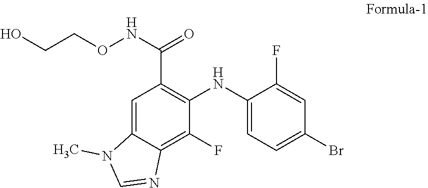 [SMILES: Cn1cnc2c(F)c(Nc3ccc(Br)cc3F)c(C(=O)NOCCO)cc21]

### RELATED APPLICATION

This application claims the benefit of priority of our Indian patents application Ser. Nos. 20/224,1000791 filed on 6 Jan. 2022, which is incorporated herein by reference.

### FIELD OF THE INVENTION

The present invention relates to an improved process for the preparation of 5-[(4-bromo-2-fluorophenyl)amino]-4-fluoro-N-(2-hydroxyethoxy)-1-methyl-1H-benzimidazole-6-carboxamide represented by the following structural formula-1, which is hereinafter referred to as Binimetinib

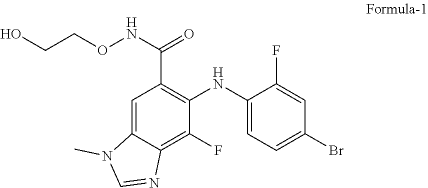 [SMILES: Cn1cnc2c(F)c(Nc3ccc(Br)cc3F)c(C(=O)NOCCO)cc21]

### BACKGROUND OF THE INVENTION

Binimetinib is an orally bioavailable, selective and potent mitogen-activated protein (MAP) kinase (MEK) 1 and MEK 2 inhibitor.

Binimetinib is approved by USFDA as MEKTOVI tablet for oral administration; in combination with Encorafenib, for the treatment of patients with unresectable or metastatic melanoma with a BRAF V600E or V600K mutation, as detected by an FDA-approved test.

U.S. Pat. No. 7,777,050 B2 (hereinafter referred as US'050) discloses Binimetinib or a pharmaceutically acceptable salt.

US'050 discloses process for the preparation of Binimetinib, comprising reducing methyl 4-amino-3-fluoro-2-((2-fluorophenyl)amino)-5-nitrobenzoate to provide methyl 4,5-diamino-3-fluoro-2-((2-fluorophenyl)amino)benzoate which upon cyclization to provide methyl 5-((2-chlorophenyl)amino)-4-fluoro-1H-benzo[d]imidazole-6-carboxylate which subsequently undergoes bromination and then methylation to provide methyl 5-((4-bromo-2-chlorophenyl)amino)-4-fluoro-1-methyl-1H-benzo[d]imidazole-6-carboxylate. Reacting the methyl 5-((4-bromo-2-chlorophenyl)amino)-4-fluoro-1-methyl-1H-benzo[d] imidazole-6-carboxylate with O-(2-vinyloxy-ethyl)-hydroxylamine to provide Binimetinib with low yield and purity.

The present inventors developed an alternate improved process for the preparation of Binimetinib. The present invention is ecofriendly, economical and provides Binimetinib with high yield and purity.

### BRIEF DESCRIPTION OF THE INVENTION

The present invention relates to an improved process for the preparation of Binimetinib.

The present invention also relates to a crystalline form of compound of formula-2 (hereinafter designated as Form-M).

The present invention also relates to a process for the preparation of crystalline form of compound of formula-2.

The present invention also relates to a process for the preparation of compound of formula-3.

### BRIEF DESCRIPTION OF DRAWINGS

FIG. 1: Illustrates the PXRD pattern of crystalline form-M of compound of formula-2.

### DETAILED DESCRIPTION OF THE INVENTION:

As used herein the term “suitable solvent” used in the present invention refers to “hydrocarbon solvents” such as n-hexane, n-heptane, cyclohexane, pet ether, toluene, pentane, cycloheptane, methyl cyclohexane, m-, o-, or p-xylene, and the like; “ether solvents” such as dimethoxy methane, tetrahydrofuran, 1,3-dioxane, 1,4-dioxane, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, dimethoxy ethane and the like; “ester solvents” such as methyl acetate, ethyl acetate, isopropyl acetate, n-butyl acetate and the like; “polar-aprotic solvents such as dimethylacetamide (DMA), dimethylformamide (DMF), dimethylsulfoxide (DMSO), N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutylketone and the like; “nitrile solvents” such as acetonitrile, propionitrile, isobutyro nitrile and the like; “alcoholic solvents” such as methanol, ethanol, n-propanol, isopropanol, n-butanol, isobutanol, t-butanol, 2-nitroethanol, 2-fluoroethanol, 2,2,2-trifluoroethanol, ethylene glycol, 2-methoxyethanol, 1,2-ethoxyethanol, diethylene glycol, 1, 2, or 3-pentanol, neo-pentyl alcohol, t-pentyl alcohol, diethylene glycol, monoethyl ether, cyclohexanol, benzyl alcohol or glycerol and the like; “polar solvents” such as water or mixtures thereof.

The “suitable base” as used in the present invention is selected from inorganic bases like “alkali metal hydroxides” such as lithium hydroxide, sodium hydroxide, potassium hydroxide and the like; “alkali metal carbonates” such as sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; ammonia; and organic bases such as “alkali metal alkoxides” such as sodium methoxide, sodium ethoxide, sodium tert-butoxide, potassium methoxide, potassium ethoxide, potassium tert-butoxide and the like; triethyl amine, methyl amine, ethylamine, 1,8-diaza bicyclo[5.4.0]undec-7-ene (DBU), 1,5-diazabicyclo(4.3.0)non-5-ene (DBN), lithiumdiiso propylamide (LDA), n-butyl lithium, tribenzylamine, isopropyl amine, diisopropylamine, diisopropylethylamine, N-methylmorpholine, N-ethylmorpholine, piperidine, dimethylamino pyridine, morpholine, pyridine, 2,6-lutidine, 2,4,6-collidine, imidazole, 1-methyl imidazole, 1,2,4-triazole, 1,4-diazabicyclo[2.2.2]octane (DABCO) or mixtures thereof.

The “suitable acid” is selected from inorganic acids such as hydrochloric acid, hydrobromic acid, hydroiodic acid, nitric acid, sulfuric acid or phosphoric acid; and organic acids such as oxalic acid, maleic acid, malonic acid, tartaric acid, fumaric acid, citric acid, malic acid, succinic acid, mandelic acid, lactic acid, acetic acid, propionic acid, 2-chloromandelate, paratoluenesulfonic acid, ethane-1,2-disulfonic acid, camphorsulfonic acid, ethane sulfonic acid, methane sulfonic acid, naphthalene-2-sulfonic acid, benzene sulfonic acid, adipic acid, glutaric acid, glutamic acid, palmitic acid or aspartic acid.

The term substituted or unsubstituted alkyl group refers to straight or branched chain hydrocarbon groups having 1-20 carbon atoms, preferably 1-7 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halo, hydroxy, cycloalkyl, alkoxy, alkenyl, alkynyl, alkylthio, alkylthiono, sulfonyl, nitro, cyano, alkoxycarbonyl, aryl, aralkoxy, heterocyclyl including indolyl, imidazolyl, furyl, thienyl, thiazolyl, pyrrolidyl, pyridyl, pyrimidyl, piperidyl, morpholinyl and the like.

In the first embodiment, the present invention provides a process for the preparation of Binimetinib of formula-1, which comprises:
a) reacting the compound of general formula-8 with compound of formula-7 in the presence of suitable base in a suitable solvent to provide compound of general formula-6;

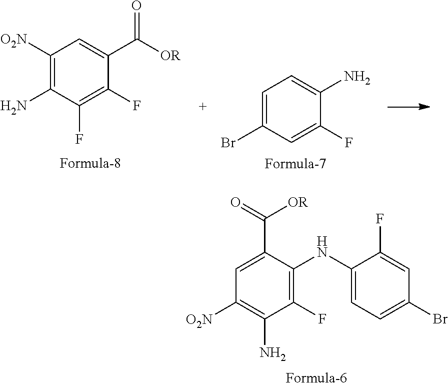

wherein “R” is selected from hydrogen, substituted or unsubstituted alkyl group.
b) reducing the compound of general formula-6 in the presence of suitable reducing agent in a suitable solvent to provide compound of general formula-5;

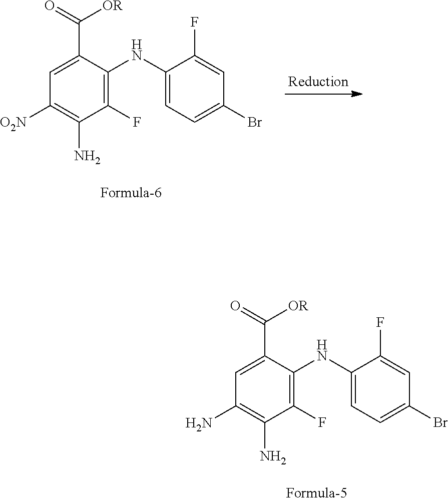

c) reacting the compound of general formula-5 with C₁₋₆ dialkoxymethane in the presence of acid in a suitable solvent to provide compound of general formula-4;

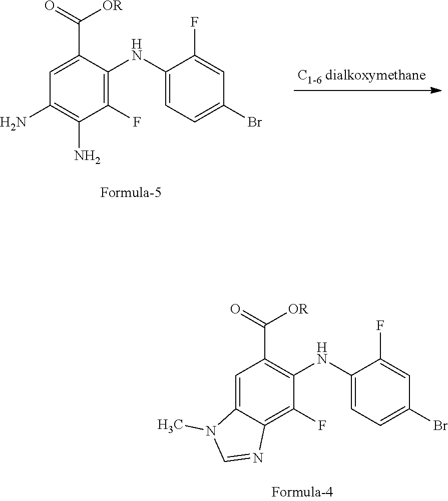

d) reacting the compound of general formula-4 with compound of formula-3 in the presence of coupling agent in a suitable base and suitable solvent to provide compound of formula-2; and

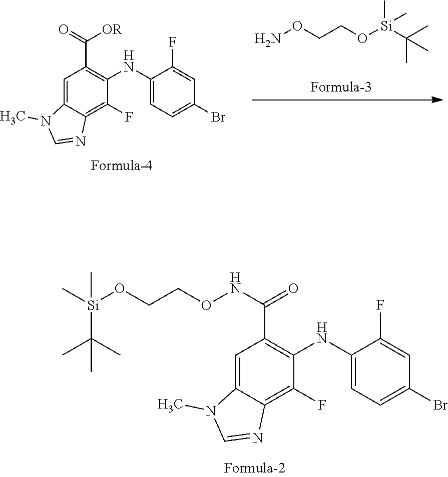

e) deprotecting the compound of formula-2 in the presence of suitable deprotecting agent in a suitable solvent to provide Binimetinib of formula-1.

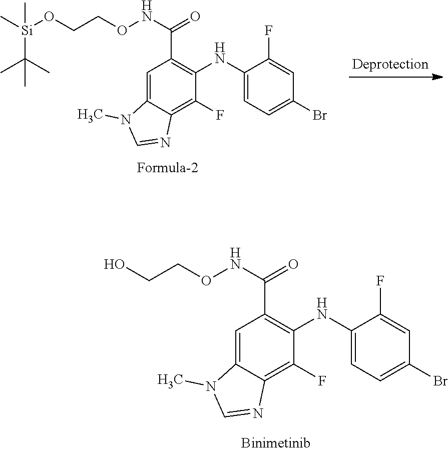 [SMILES: Cn1cnc2c(F)c(Nc3ccc(Br)cc3F)c(C(=O)NOCCO[Si](C)(C)C(C)(C)C)cc21 ; Cn1cnc2c(F)c(Nc3ccc(Br)cc3F)c(C(=O)NOCCO)cc21]

In the process of the first embodiment, the suitable solvent used in steps a) to e) is selected from alcohol solvents, ester solvents, hydrocarbon solvents, nitrile solvents, polar aprotic solvents, ketone solvents, ether solvents, chloro solvents, and water or mixture thereof; the suitable base used in step-a) and step-d) is selected from inorganic base or organic base; the suitable acid used in step-c) is selected from inorganic acid or organic acid.

In the process of the first embodiment, the suitable reducing agent used in step-b) is selected from Pd/C, Pt/C, PtO₂, Pd(OH)₂, Nickel, Raney nickel, Rhodium, sodium dithionate, sodium amalgam, Fe, Fe in acidic media like NH₄Cl or HCl or acetic acid, Sn in acidic media like HCl, Zn dust, Zn in acidic media like HCl or NH₄Cl or acetic acid and the like.

In the process of the first embodiment, the suitable C₁₋₆ dialkoxymethane used in step-c) is selected from diethoxymethane or dimethoxymethane. In the process of the first embodiment, the suitable coupling agent used in step-d) is selected from PyBOP, BOP, TBTU, EDCI, HATU, HBTU, HCTU, DCC, CDI, DIC, isobutylchloroformate, pivaloyl chloride, oxalyl chloride, thionyl chloride, 1-propanephosphonic acid, EDC·HCl, HOBt, DMAP, HOAt, HOCt, HOSu, BOMI, BDMP, BMPI or CMPI and the like.

In the process of the first embodiment, the suitable deprotecting agent used in step-e) is selected from HCl, TFA, phosphoric acid, sulfuric acid, trimethylsilylchloride, p-toluenesulfonic acid and the like.

In the first aspect of the first embodiment, the present invention provides a process for the preparation of Binimetinib of formula-1, which comprises:
a) reacting the compound of formula-8a with compound of formula-7 in the presence of N,N-diisopropylethylamine in water to provide compound of formula-6a;

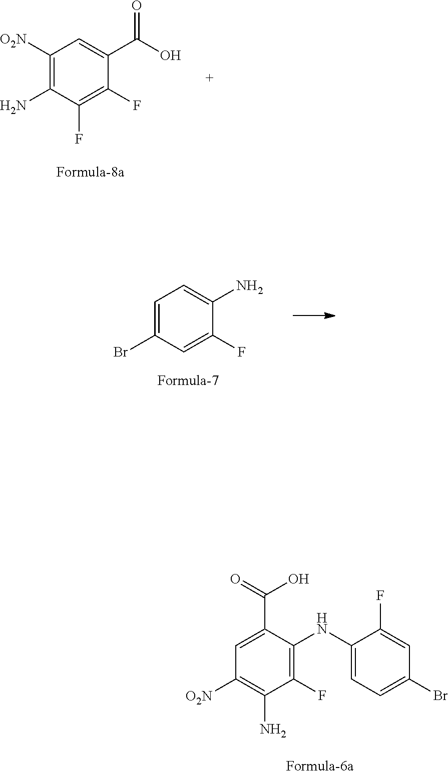 [SMILES: Nc1c([N+](=O)[O-])cc(C(=O)O)c(F)c1F ; Nc1ccc(Br)cc1F ; Nc1c([N+](=O)[O-])cc(C(=O)O)c(Nc2ccc(Br)cc2F)c1F]

b) reducing the compound of formula-6a in the presence of Raney Ni in methanol and tetrahydrofuran to provide compound of formula-5a;

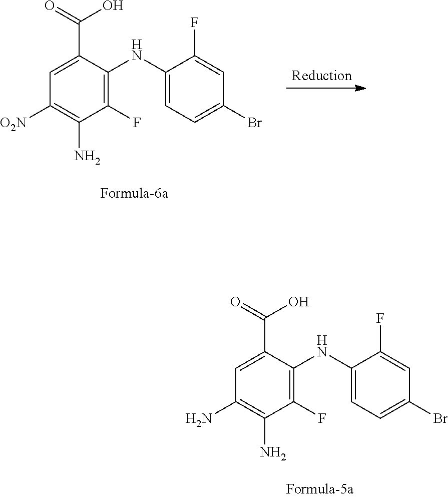 [SMILES: Nc1c([N+](=O)[O-])cc(C(=O)O)c(Nc2ccc(Br)cc2F)c1F ; Nc1cc(C(=O)O)c(Nc2ccc(Br)cc2F)c(F)c1N]

1c) reacting the compound of formula-5a with diethoxymethane in the presence of p-toluenesulfonic acid in acetonitrile and water to provide compound of formula-4a;

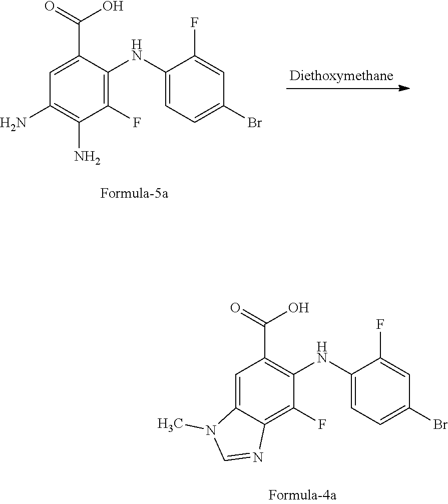 [SMILES: Nc1cc(C(=O)O)c(Nc2ccc(Br)cc2F)c(F)c1N ; Cn1cnc2c(F)c(Nc3ccc(Br)cc3F)c(C(=O)O)cc21]

d) reacting the compound of formula-4a with compound of formula-3 in the presence of HOBT and EDC·HCl in N,N-diisopropylethylamine and dimethylformamide to provide compound of formula-2; and

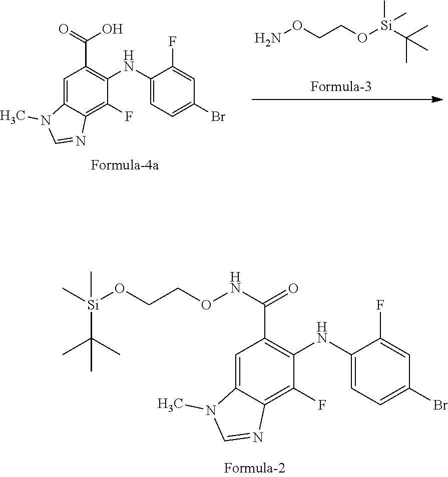 [SMILES: Cn1cnc2c(F)c(Nc3ccc(Br)cc3F)c(C(=O)O)cc21 ; CC(C)(C)[Si](C)(C)OCCON ; Cn1cnc2c(F)c(Nc3ccc(Br)cc3F)c(C(=O)NOCCO[Si](C)(C)C(C)(C)C)cc21]

e) deprotecting the compound of formula-2 in the presence of TFA in dichloromethane to provide Binimetinib of formula-1.

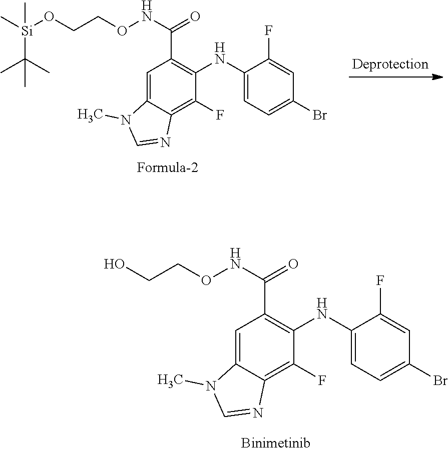 [SMILES: Cn1cnc2c(F)c(Nc3ccc(Br)cc3F)c(C(=O)NOCCO[Si](C)(C)C(C)(C)C)cc21 ; Cn1cnc2c(F)c(Nc3ccc(Br)cc3F)c(C(=O)NOCCO)cc21]

In the second embodiment, the present invention provides a compound of formula-2.

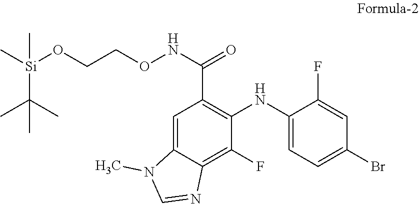 [SMILES: Cn1cnc2c(F)c(Nc3ccc(Br)cc3F)c(C(=O)NOCCO[Si](C)(C)C(C)(C)C)cc21]

The compound of formula-2 is useful in the preparation of Binimetinib of formula-1.

In the third embodiment, the present invention provides a crystalline form of compound of formula-2.

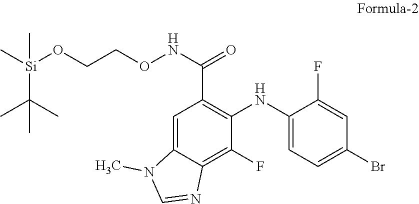 [SMILES: Cn1cnc2c(F)c(Nc3ccc(Br)cc3F)c(C(=O)NOCCO[Si](C)(C)C(C)(C)C)cc21]

In the fourth embodiment, the present invention provides a crystalline Form-M of compound of formula-2.

In the fourth embodiment, the crystalline form-M of compound of formula-2 is characterized by its X-ray powder diffraction (XRD) pattern having peaks at about 6.3, 9.1 and 11.8±0.2 degrees 2-theta.

In the fourth embodiment, the crystalline form-M of compound of formula-2 is further characterized by its X-ray powder diffraction (XRD) pattern having peaks at about 5.0, 11.2, 13.8, 18.2, 18.7 and 23.9±0.2 degrees of 2-theta.

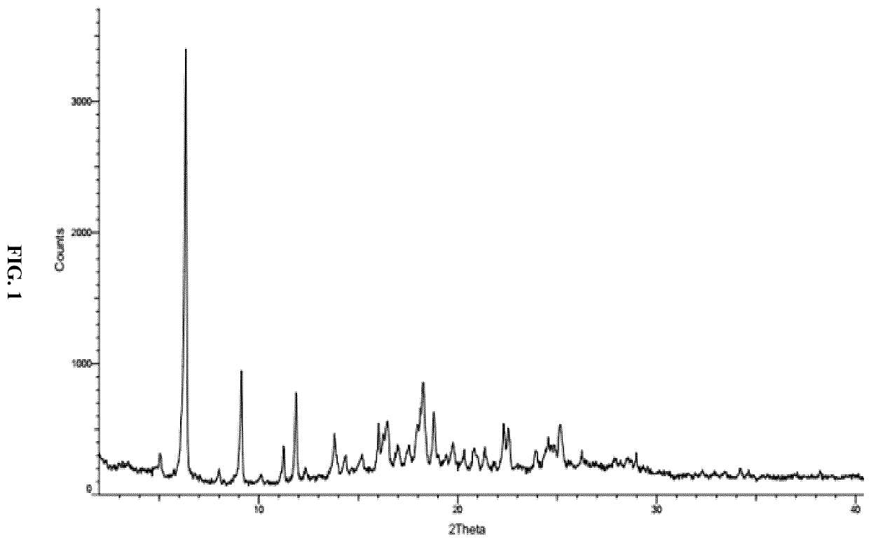

In the fourth embodiment, the crystalline form-M of compound of formula-2 is further characterized by its X-ray powder diffraction (XRD) pattern as illustrated in FIG. 1.

In the fourth embodiment, the crystalline form-M of compound of formula-2 is useful in the preparation of pure Binimetinib.

In the fifth embodiment, the present invention provides a process for the preparation of crystalline Form-M of compound of formula-2, which comprises reacting compound of formula-4a with compound of formula-3 in a suitable solvent and isolating the crystalline Form-M of compound of formula-2.

In the process of the fifth embodiment, a suitable solvent used for the preparation of crystalline Form-M of compound of formula-2 is selected from alcohol solvents, ester solvents, chloro solvents, nitrile solvents, ether solvents, ketone solvents, hydrocarbon solvents, polar aprotic solvents and water or mixture thereof.

In the process of the fifth embodiment, isolation of crystalline Form-M of compound of formula-2 can be carried out by any methods known in the art or crystalline Form-M of compound of formula-2 can be isolated by employing any of the techniques, but not limited to: decantation, filtration by gravity or suction, evaporation or distillation with or without vacuum, aqueous thin film drying, centrifugation, adding solvent to make slurry followed by filtration, or other techniques specific to the equipment used and the like, and optionally washing with a solvent.

In the process of the fifth embodiment, drying the crystalline Form-M of compound of formula-2 can be carried out in a suitable drying equipment such as tray dryer, vacuum oven, rotatory cone dryer, air oven, fluidized bed dryer, spin flash dryer, flash dryer, or the like. The drying can be carried out at atmospheric pressure or under reduced pressures at temperatures of less than about 100° C., less than about 60° C., less than about 40° C., or any other suitable temperatures. The drying can be carried out for any time period required for obtaining a desired quality, such as from about 15 minutes to 10 hours or longer.

In the sixth embodiment, the present invention provides a process for the preparation of compound of formula-2, which comprises reacting compound of formula-4a with compound of formula-3 to provide compound of formula-2.

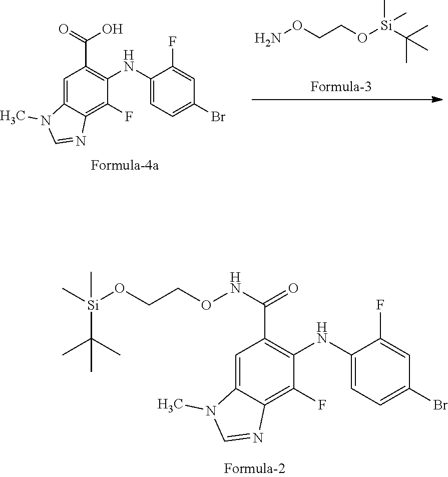 [SMILES: Cn1cnc2c(F)c(Nc3ccc(Br)cc3F)c(C(=O)O)cc21 ; CC(C)(C)[Si](C)(C)OCCON ; Cn1cnc2c(F)c(Nc3ccc(Br)cc3F)c(C(=O)NOCCO[Si](C)(C)C(C)(C)C)cc21]

In the seventh embodiment, the present invention provides a process for the preparation of Binimetinib, which comprises deprotecting the compound of formula-2 to provide Binimetinib of formula-1.

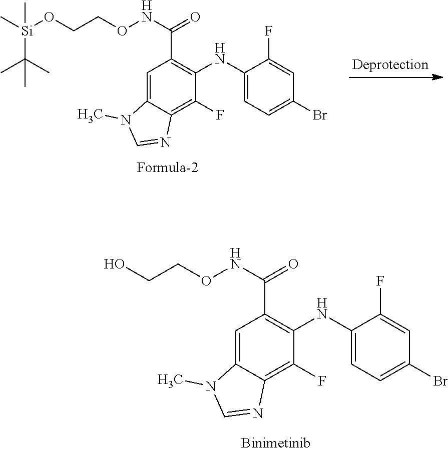 [SMILES: Cn1cnc2c(F)c(Nc3ccc(Br)cc3F)c(C(=O)NOCCO[Si](C)(C)C(C)(C)C)cc21 ; Cn1cnc2c(F)c(Nc3ccc(Br)cc3F)c(C(=O)NOCCO)cc21]

In the eighth embodiment, the present invention provides a process for the preparation of compound of formula-3, which comprises:
a) mesylation of compound of formula-11 to provide compound of formula-10;

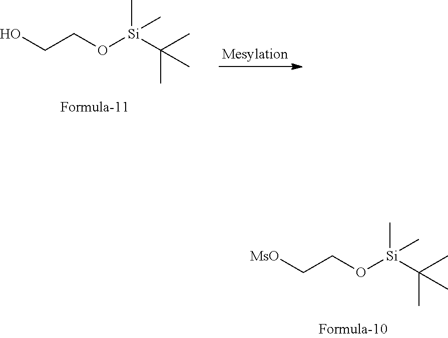

b) reacting the compound of formula-10 with N-hydroxyphthalimide to provide compound of formula-9; and

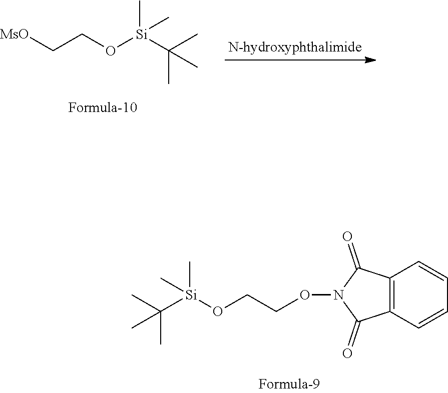 [SMILES: CC(C)(C)[Si](C)(C)OCCOS(C)(=O)=O ; CC(C)(C)[Si](C)(C)OCCON1C(=O)c2ccccc2C1=O]

c) converting the compound of formula-9 to compound of formula-3.

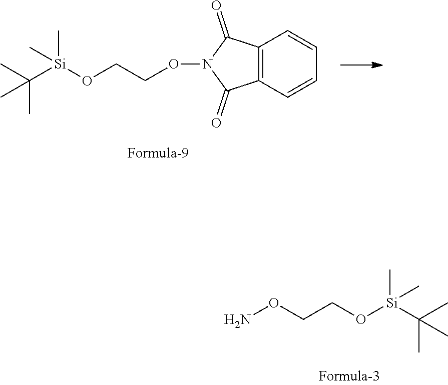 [SMILES: CC(C)(C)[Si](C)(C)OCCON1C(=O)c2ccccc2C1=O ; CC(C)(C)[Si](C)(C)OCCON]

In the ninth embodiment, the present invention provides a compound of general formula-12, which is useful for the synthesis of compound of formulae 3, 2 and Binimetinib.

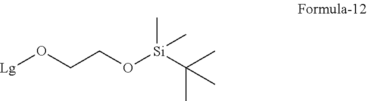

wherein, “Lg” is a leaving group such as tosyl, mesyl, acyl, benzyl and like.

The compounds of formulae 7, 8 and 11 used in the present invention are synthesized from any of the known prior art processes.

Binimetinib obtained according to the present invention can be purified using a suitable solvent selected form alcohol solvents, ester solvents, hydrocarbon solvents, nitrile solvents, ketone solvents, ether solvents, chloro solvents, and water or mixture thereof.

Binimetinib produced by the present invention can be further micronized or milled in conventional techniques to get the desired particle size to achieve desired solubility profile based on different forms of pharmaceutical composition requirements. Techniques that may be used for particle size reduction include, but not limited to ball milling, roller milling and hammer milling, and jet mills. Milling or micronization may be performed before drying, or after the completion of drying of the product.

The invention also encompasses pharmaceutical compositions comprising Binimetinib of the present invention. As used herein, the term “pharmaceutical compositions” or “pharmaceutical formulations” include tablets, pills, powders, liquids, suspensions, emulsions, granules, capsules, suppositories, or injection preparations.

### P-XRD Method of Analysis

PXRD analysis of compounds produced by the present invention were carried out using BRUKER D8 ADVANCE/AXS X-Ray diffractometer using Cu Kα radiation of wavelength 1.5406 A° and continuous scan speed of 0.03°/min.

The process described in the present invention is demonstrated in examples illustrated below. These examples are provided as illustration only and therefore should not be construed as limitation of the scope of the invention.

### Examples

### Example-1: Preparation of 2-(2-(tert-butyldimethylsilyloxy)ethoxy)isoindoline-1,3-dione of Formula-9

Methyl tert-butyl ether (400.0 ml) was added to 2-(tert-butyldimethylsilyloxy)ethanol (40.0 gm) and triethylamine (34.4 gm) at 25-30° C. and stirred for 5 minutes. Cooled the mixture to 0-5° C. and stirred for 15 minutes. Methanesulfonyl chloride (28.5 gm) was added to the mixture at 0-5° C. and stirred for 1 hour. Raised the temperature of the mixture to 25-30° C. and stirred for 2 hours. Sodium bicarbonate solution was slowly added to the mixture at 25-30° C. and stirred for 1 hour. Layers were separated and extracted the aqueous layer with methyl tert-butyl ether. Combined the organic layers and distilled off the solvent completely from the organic layer.

Dimethylformamide (200.0 ml), N-hydroxyphthalimide (33.6 gm) and 1,8-Diazabicyclo[5.4.0]undec-7-ene (34.4 gm) were added to the above obtained compound at 25-30° C. and stirred for 10 minutes. Heated the mixture to 80-85° C. and stirred for 5 hours. Cooled the mixture to 25-30° C. and stirred for 30 minutes. The mixture was slowly added to the pre-cooled water (800.0 ml) at 0-5° C. and stirred for 30 minutes. Raised the temperature of the mixture to 25-30° C. and stirred for 90 minutes. Filtered the solid, washed with water and dried to get the title compound. Yield: 48.0 gm.

### Example-2: Preparation of O-(2-(tert-butyldimethylsilyloxy) ethyl) hydroxylamine of Formula-3

Dichloromethane (270.0 ml) and methanol (270.0 ml) were added to compound of formula-9 (45.0 gm) at 25-30° C. and stirred for 15 minutes. Cooled the mixture to 10-15° C. and stirred for 40 minutes. Hydrazine hydrate (35.4 gm) was added to the mixture at 10-15° C. and stirred for 5 hours. Filtered the mixture and washed with dichloromethane. Water (135.0 ml) was added to the filtrate at 25-30° C. and stirred for 15 minutes. Layers were separated and extracted the aqueous layer with dichloromethane. Combined the organic layers and washed with sodium chloride solution. Distilled off the solvent completely from the organic layer. n-Heptane (225.0 ml) was added to the obtained compound at 25-30° C. Cooled the mixture to 10-15° C. and stirred for 1 hour. Filtered the mixture and washed with n-heptane. Distilled off the solvent completely from the filtrate to get the title compound. Yield: 22.0 gm.

### Example-3: Preparation of 4-amino-2,3-difluoro-5-nitrobenzoic acid of Formula-8a

Water (150.0 ml) was added to 2,3,4-trifluoro-5-nitro-benzoic acid (50.0 gm) at 25-30° C. and stirred for 20 minutes. Cooled the mixture to 0-5° C. and stirred for 25 minutes. Ammonia (117.5 ml) was added to the mixture at 0-5° C. and stirred for 30 minutes. Raised the temperature of the mixture to 25-30° C. and stirred for 2 hours. Cooled the mixture to 10-15° C. and stirred for 20 minutes. Mixture pH was adjusted with HCl. Raised the temperature of the mixture to 25-30° C. and stirred for 2 hours. Filtered the solid, washed with water and dried to get the title compound. Yield: 46.0 gm.

### Example-4: Preparation of 4-amino-2-((4-bromo-2-fluorophenyl)amino)-3-fluoro-5-nitrobenzoic acid of Formula-6a

N,N-Diisopropylethylamine (45.3 gm) was added to compound of formula-8a (45.0 gm) and 4-bromo-2-fluoroaniline (117.6 gm) at 25-30° C. and stirred for 10 minutes. Heated the mixture to 105-110° C. and stirred for 8 hours. Cooled the mixture to 25-30° C. and stirred for 1 hour. Water (450.0 ml) was added to the mixture at 25-30° C. Cooled the mixture to 10-15° C. and stirred for 30 minutes. Mixture pH was adjusted with acetic acid solution to 4-5. Raised the temperature of the mixture to 25-30° C. and stirred for 1 hour. Filtered the solid and washed with water. Acetonitrile (225.0 ml) was added to the obtained compound at 25-30° C. and stirred for 10 minutes. Heated the mixture to 45-50° C. and stirred for 1 hour. Filtered the solid, washed with acetonitrile and dried to get the title compound. Yield: 46.0 gm.

### Example-5: Preparation of 4,5-diamino-2-((4-bromo-2-fluorophenyl)amino)-3-fluorobenzoic Acid of Formula-5a

Methanol (337.5 ml) and tetrahydrofuran (337.5 ml) were added to compound of formula-6a (45.0 gm) at 25-30° C. in an autoclave vessel. Raney Ni (2.25 gm) and methanol (20.0 ml) slurry were added to the mixture at 25-30° C. and closed the vessel. Flushed the mixture with nitrogen gas at 25-30° C. 3.0 kg/cm hydrogen gas pressure was applied to the mixture at 25-30° C. Heated the mixture to 65-70° C. and stirred for 3 hours. 3.0 kg/cm hydrogen gas pressure was applied to the mixture at 65-70° C. and stirred for 10 hours. Cooled the mixture to 25-30° C. and stirred for 30 minutes. Released the pressure at 25-30° C. Filtered the mixture through hyflow bed and washed the hyflow bed with tetrahydrofuran and methanol. Distilled off the solvent completely from the filtrate. n-Heptane (45.0 ml) was added to the obtained compound at 25-30° C. and stirred for 80 minutes. Filtered the solid, washed with n-heptane and dried to get the title compound. Yield: 36.0 gm.

### Example-6: Preparation of 5-((4-bromo-2-fluorophenyl)amino)-4-fluoro-1-methyl-1H-benzo[d]imidazole-6-carboxylic Acid of Formula-4a

Acetonitrile (350.0 ml) was added to compound of formula-5a (35.0 gm) at 25-30° C. and stirred for 10 minutes. p-Toluenesulfonic acid (37.0 gm) and water (3.5 ml) were added to the mixture at 25-30° C. and stirred for 5 minutes. Diethoxymethane (24.5 gm) was added to the mixture at 25-30° C. and stirred for 20 minutes. Heated the mixture to 60-65° C. and stirred for 2 hours. Cooled the mixture to 25-30° C. and stirred for 90 minutes. Filtered the solid and washed with acetonitrile. Water (350.0 ml) was added to the obtained compound at 25-30° C. and stirred for 30 minutes. Cooled the mixture to 15-20° C. Mixture pH was adjusted with liquor ammonia. Raised the temperature of the mixture to 25-30° C. and stirred for 50 minutes. Filtered the solid, washed with water and dried to get the title compound. Yield: 23.0 gm.

### Example-7: Preparation of Crystalline Form-M of 5-((4-bromo-2-fluorophenyl)amino)-N-(2-((tert-butyldimethylsilyl)oxy)ethoxy)-4-fluoro-1-methyl-1H-benzo[d]imidazole-6-carboxamide of Formula-2

Dimethylformamide (176.0 ml) was added to compound of Formula-4a (22.0 gm), compound of formula-3 (14.30 gm) and hydroxybenzotriazole (2.33 gm) at 25-30° C. and stirred for 20 minutes. Diisopropylethylamine (9.30 gm) was added to the mixture at 25-30° C. and stirred for 10 minutes. EDC·HCl (13.79 gm) was added to the mixture for five lots at 25-30° C. and stirred for 5 hours. To the mixture was slowly added to the water (880.0 ml) at 25-30° C. and stirred for 90 minutes. Filtered the solid, washed with water and dried. Isopropanol (200.0 ml) was added to the obtained compound at 25-30° C. and stirred for 15 minutes. Heated the mixture to 80-85° C. and stirred for 1 hour. Activated carbon was added to the mixture at 80-85° C. and stirred for 15 minutes. Filtered the mixture through hyflow bed and washed the hyflow bed with isopropanol. Distilled off the solvent completely from the filtrate. Isopropanol (56.0 ml) and acetonitrile (19.0 ml) were added to the obtained compound at 50-55° C. and stirred for 10 minutes. Heated the mixture to 75-80° C. and stirred for 10 minutes. Cooled the mixture to 25-30° C. and stirred for 90 minutes. Methyl tert-butyl ether (44.0 ml) was added to the mixture at 25-30° C. and stirred for 20 minutes. Cooled the mixture to 10-15° C. and stirred for 2 hours. Filtered the solid, washed with methyl tert-butyl ether and dried to get the title compound. Yield: 16.0 gm; The P-XRD pattern of the obtained compound is illustrated in FIG. 1.

### Example-8: Preparation of Binimetinib of Formula-1

Dichloromethane (150.0 ml) was added to compound of formula-2 (15.0 gm) at 25-30° C. and stirred for 5 minutes. Cooled the mixture to 5-10° C. and stirred for 20 minutes. Trifluoroacetic acid (75.0 ml) was added to the mixture at 5-10° C. and stirred for 1 hour. Raised the temperature of the mixture to 25-30° C. and stirred for 4 hours. Mixture was added to pre-cooled sodium carbonate solution at 5-10° C. and stirred for 1 hour. Raised the temperature of the mixture to 25-30° C. and stirred for 1 hour. Filtered the solid and washed with water. Water (300.0 ml) was added to the obtained compound at 25-30° C. and stirred for 10 minutes. Heated the mixture to 55-60° C. and stirred for 90 minutes. Filtered the solid, washed with water and dried. Acetonitrile (220.0 ml) was added to the obtained compound at 25-30° C. and stirred for 5minutes. Heated the mixture to 80-85° C. and stirred for 1 hour. Cooled the mixture to 25-30° C. and stirred for 2 hours. Filtered the solid and washed with acetonitrile. Acetonitrile (200.0 ml) was added to the obtained compound at 25-30° C. and stirred for 5 minutes. Heated the mixture to 80-85° C. and stirred for 1 hour. Cooled the mixture to 25-30° C. and stirred for 2 hours. Filtered the solid, washed with acetonitrile and dried to get the title compound. Yield: 9.0 gm; Purity by HPLC: 99.59%.

We claim:

1. A process for the preparation of compound of formula-2, which comprises reacting compound of formula-4a with compound of formula-3 to provide compound of formula-2.

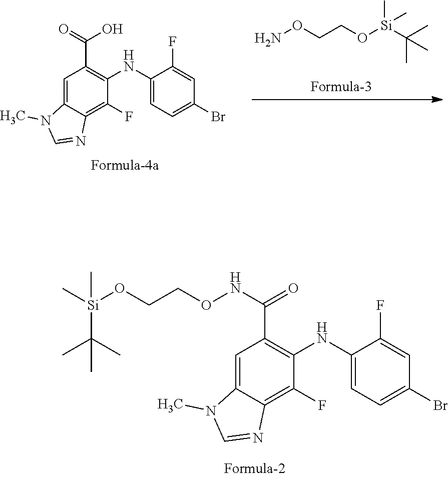 [SMILES: Cn1cnc2c(F)c(Nc3ccc(Br)cc3F)c(C(=O)O)cc21 ; CC(C)(C)[Si](C)(C)OCCON ; Cn1cnc2c(F)c(Nc3ccc(Br)cc3F)c(C(=O)NOCCO[Si](C)(C)C(C)(C)C)cc21]

2. The process as claimed in claim 1 wherein, the process comprises reacting compound of formula-4a with compound of formula-3 in the presence of coupling agent in a base and a solvent to provide compound of formula-2.

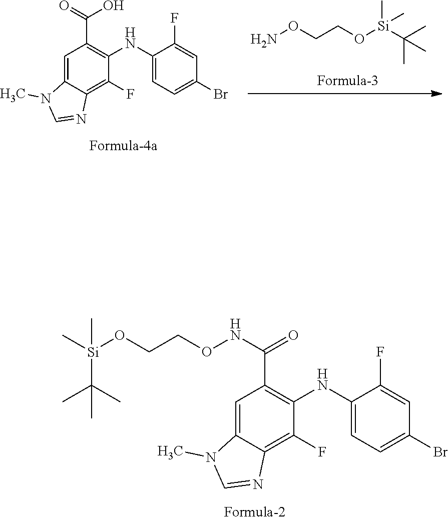 [SMILES: Cn1cnc2c(F)c(Nc3ccc(Br)cc3F)c(C(=O)O)cc21 ; CC(C)(C)[Si](C)(C)OCCON ; Cn1cnc2c(F)c(Nc3ccc(Br)cc3F)c(C(=O)NOCCO[Si](C)(C)C(C)(C)C)cc21]

3. The process as claimed in claim 2 wherein, the coupling agent is selected from PyBOP, BOP, TBTU, EDCI, HATU, HBTU, HCTU, DCC, CDI, DIC, isobutylchloroformate, pivaloyl chloride, oxalyl chloride, thionyl chloride, 1-propanephosphonic acid, EDC·HCl, HOBt, DMAP, HOAt, HOCt, HOSu, BOMI, BDMP, BMPI or CMPI and the like; the base is selected from inorganic base or organic base; the solvent is selected from alcohol solvents, ester solvents, hydrocarbon solvents, nitrile solvents, polar aprotic solvents, ketone solvents, ether solvents, chloro solvents, and water or mixture thereof;

4. A process for the preparation of Binimetinib, which comprises deprotecting the compound of formula-2 in the presence of deprotecting agent to provide Binimetinib of formula-1.

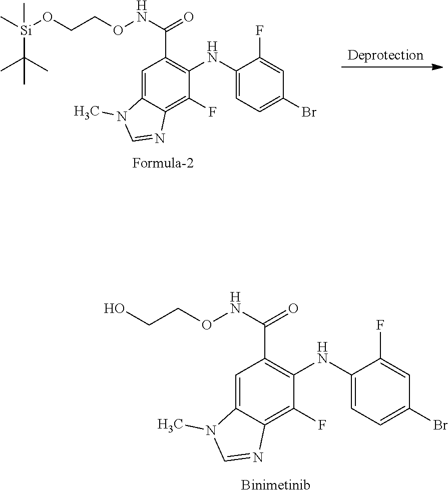 [SMILES: Cn1cnc2c(F)c(Nc3ccc(Br)cc3F)c(C(=O)NOCCO[Si](C)(C)C(C)(C)C)cc21 ; Cn1cnc2c(F)c(Nc3ccc(Br)cc3F)c(C(=O)NOCCO)cc21]

5. The process as claimed in claim 4 wherein, the deprotecting agent is selected from hydrochloric acid, trifluoroacetic acid, phosphoric acid, sulfuric acid, trimethylsilylchloride, p-toluenesulfonic acid and the like; the solvent is selected from alcohol solvents, ester solvents, hydrocarbon solvents, nitrile solvents, polar aprotic solvents, ketone solvents, ether solvents, chloro solvents, and water or mixture thereof.

6. A compound of formula-2.

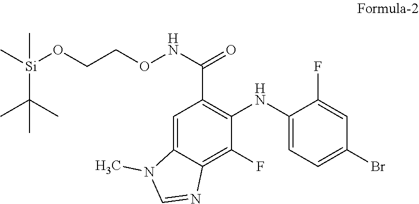 [SMILES: Cn1cnc2c(F)c(Nc3ccc(Br)cc3F)c(C(=O)NOCCO[Si](C)(C)C(C)(C)C)cc21]

7. A process using a compound of formula-2 for preparing Binimetinib.

8. Crystalline compound of formula-2.

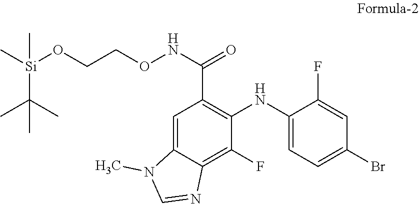 [SMILES: Cn1cnc2c(F)c(Nc3ccc(Br)cc3F)c(C(=O)NOCCO[Si](C)(C)C(C)(C)C)cc21]

9. A crystalline form-M of compound of formula-2 is characterized by its X-ray powder diffractogram (XRD) having peaks at about 6.3, 9.1 and 11.8±0.2 degrees 2-theta.

10. The crystalline Form-M of compound of formula-2 as claimed in claim 9 is further characterized by the X-ray powder diffractogram (XRD) pattern as illustrated in FIG. 1.

11. A process for the preparation of crystalline Form-M of compound of formula-2, which comprises;
a) reacting compound of formula-4a with compound of formula-3 in a solvent; and
b) isolating the crystalline Form-M of compound of formula-2.

12. The process as claimed in claim 11 wherein, the solvent used in step-a) is selected from alcohol solvents, ester solvents, chloro solvents, nitrile solvents, ether solvents, ketone solvents, hydrocarbon solvents, polar aprotic solvents and water or mixture thereof.

13. A process for the preparation of Binimetinib of formula-1, which comprises:
a) reacting the compound of general formula-8 with compound of formula-7 in the presence of a base in a solvent to provide compound of general formula-6; 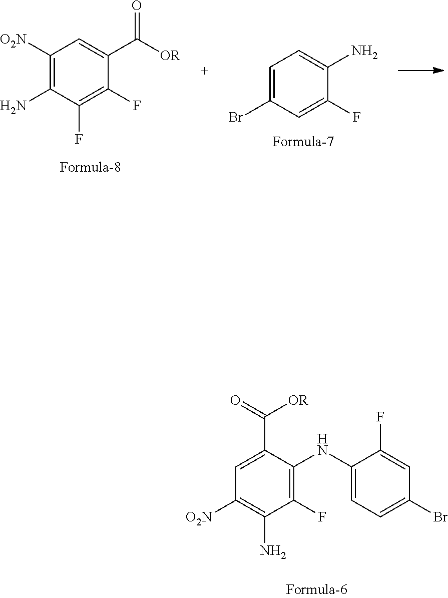
wherein “R” is selected from hydrogen, substituted or unsubstituted alkyl group.
b) reducing the compound of general formula-6 in the presence of reducing agent in a solvent to provide general compound of formula-5; 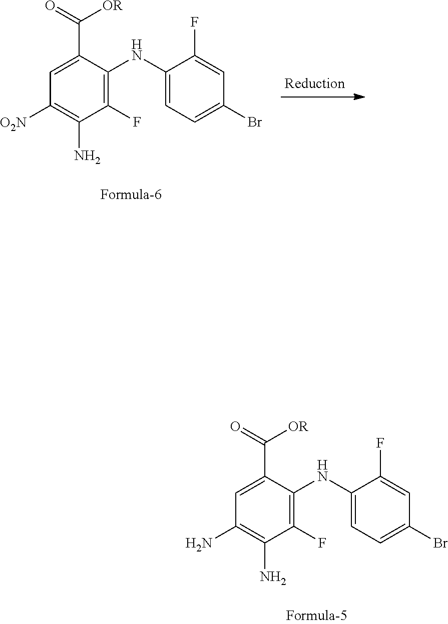
c) reacting the compound of general formula-5 with C₁₋₆ dialkoxymethane in the presence of acid in a solvent to provide compound of general formula-4; 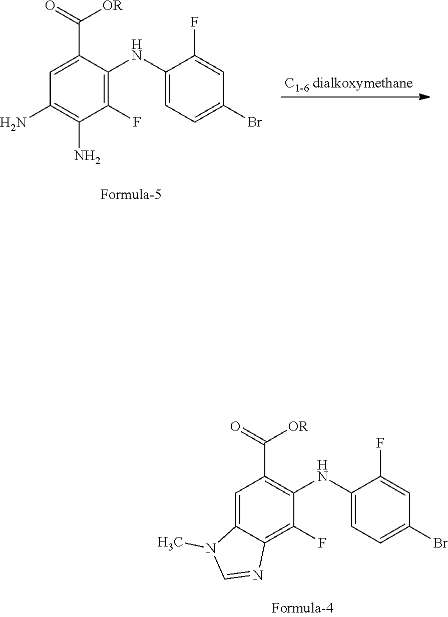
d) reacting compound of general formula-4 with compound of formula-3 in the presence of coupling agent in a base and a solvent to provide compound of formula-2; and 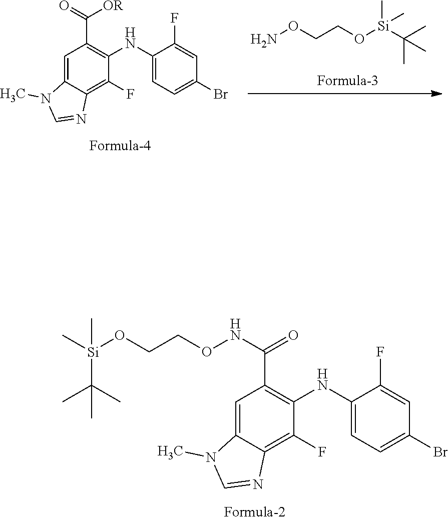
e) deprotecting the compound of formula-2 in the presence of deprotecting agent in a solvent to provide Binimetinib of formula-1.

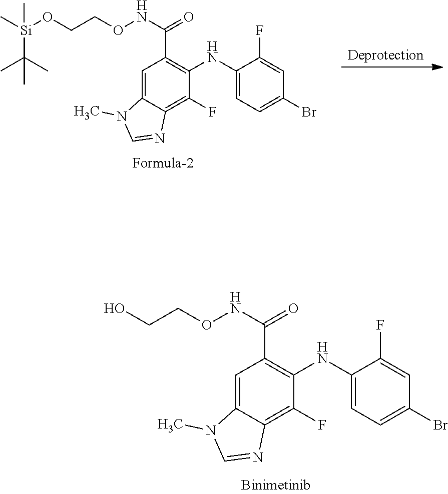 [SMILES: Cn1cnc2c(F)c(Nc3ccc(Br)cc3F)c(C(=O)NOCCO[Si](C)(C)C(C)(C)C)cc21 ; Cn1cnc2c(F)c(Nc3ccc(Br)cc3F)c(C(=O)NOCCO)cc21]

14. The process as claimed in claim 13 wherein, the solvent used in steps a) to e) is selected from alcohol solvents, ester solvents, hydrocarbon solvents, nitrile solvents, polar aprotic solvents, ketone solvents, ether solvents, chloro solvents, and water or mixture thereof; the base used in step-a) and step-d) is selected from inorganic base or organic base; the acid used in step-c) is selected from inorganic acid or organic acid; the reducing agent used in step-b) is selected from Pd/C, Pt/C, PtO₂, Pd(OH)₂, Nickel, Raney nickel, Rhodium, sodium dithionate, sodium amalgam, Fe, Fe in acidic media like NH₄Cl or HCl or acetic acid, Sn in acidic media like HCl, Zn dust, Zn in acidic media like HCl or NH₄Cl or acetic acid and the like; the coupling agent used in step-d) is selected from PyBOP, BOP, TBTU, EDCI, HATU, HBTU, HCTU, DCC, CDI, DIC, isobutylchloroformate, pivaloyl chloride, oxalyl chloride, thionyl chloride, 1-propanephosphonic acid, EDC·HCl, HOBt, DMAP, HOAt, HOCt, HOSu, BOMI, BDMP, BMPI or CMPI and the like; the deprotecting agent used in step-e) is selected from HCl, TFA, phosphoric acid, sulfuric acid, trimethylsilylchloride, p-toluenesulfonic acid and the like.

15. The process as claimed in claim 13 wherein, the C₁₋₆ dialkoxymethane used in step-c) is selected from diethoxymethane or dimethoxymethane

16. The process as claimed in claim 13 wherein, the process comprises:
a) reacting the compound of formula-8a with compound of formula-7 in the presence of N,N-diisopropylethylamine in water to provide compound of formula-6a; 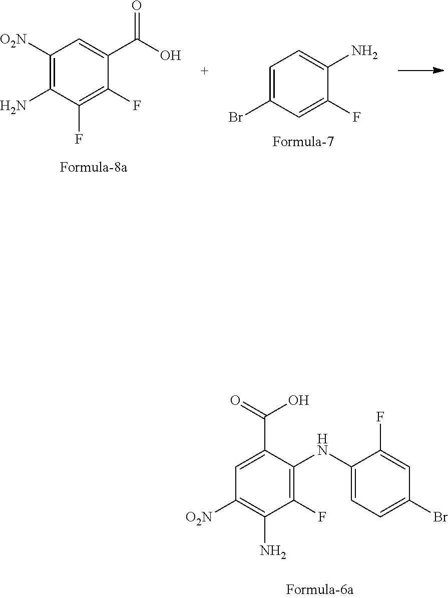 [SMILES: Nc1c([N+](=O)[O-])cc(C(=O)O)c(F)c1F ; Nc1ccc(Br)cc1F ; Nc1c([N+](=O)[O-])cc(C(=O)O)c(Nc2ccc(Br)cc2F)c1F]
b) reducing the compound of formula-6a in the presence of Raney Ni in methanol and tetrahydrofuran to provide compound of formula-5a; 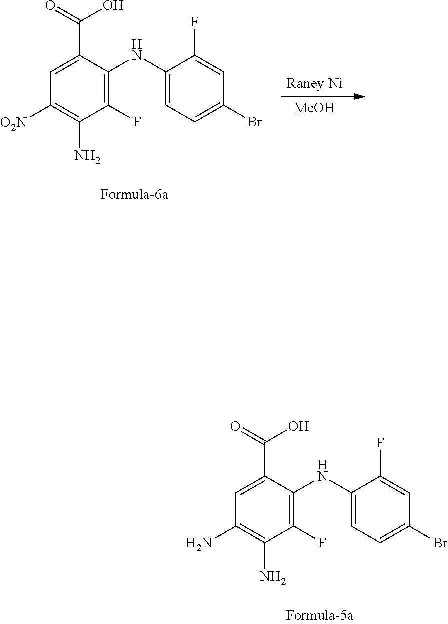 [SMILES: "MeOH" ; Nc1c([N+](=O)[O-])cc(C(=O)O)c(Nc2ccc(Br)cc2F)c1F ; Nc1cc(C(=O)O)c(Nc2ccc(Br)cc2F)c(F)c1N]
c) reacting the compound of formula-5a with diethoxymethane in the presence of p-toluenesulfonic acid in acetonitrile and water to provide compound of formula-4a; 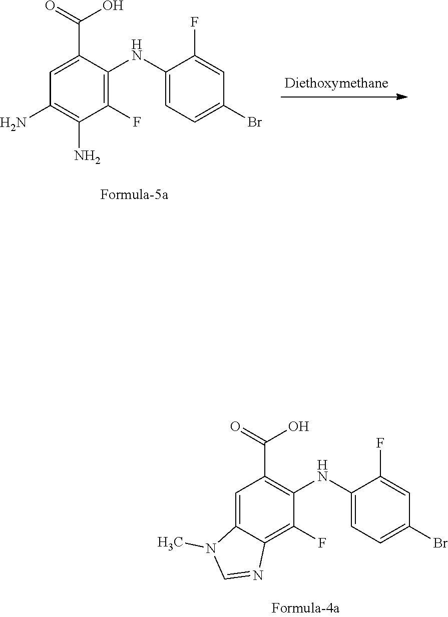 [SMILES: Nc1cc(C(=O)O)c(Nc2ccc(Br)cc2F)c(F)c1N ; Cn1cnc2c(F)c(Nc3ccc(Br)cc3F)c(C(=O)O)cc21]
d) reacting the compound of formula-4a with compound of formula-3 in the presence of HOBT and EDC·HCl in N,N-diisopropylethylamine and dimethylformamide to provide compound of formula-2; and 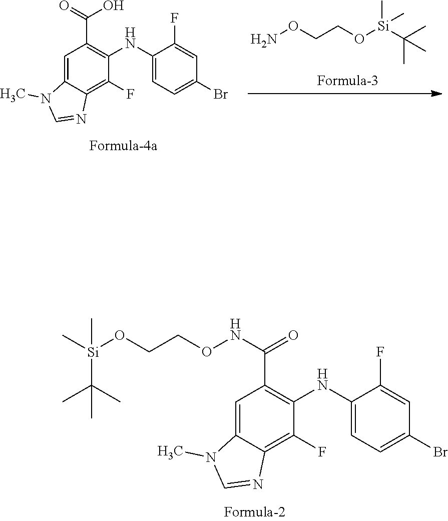 [SMILES: Cn1cnc2c(F)c(Nc3ccc(Br)cc3F)c(C(=O)O)cc21 ; CC(C)(C)[Si](C)(C)OCCON ; Cn1cnc2c(F)c(Nc3ccc(Br)cc3F)c(C(=O)NOCCO[Si](C)(C)C(C)(C)C)cc21]
e) deprotecting the compound of formula-2 in the presence of Trifluoroacetic acid in dichloromethane to provide Binimetinib of formula-1.

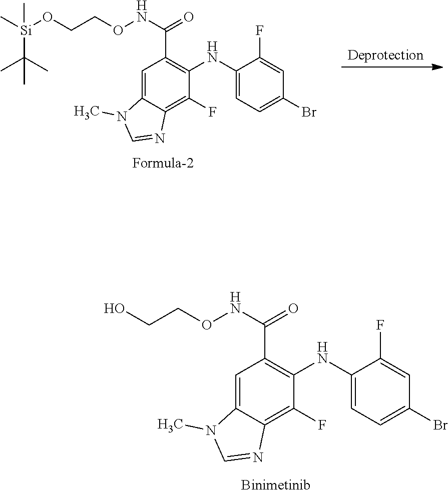 [SMILES: Cn1cnc2c(F)c(Nc3ccc(Br)cc3F)c(C(=O)NOCCO[Si](C)(C)C(C)(C)C)cc21 ; Cn1cnc2c(F)c(Nc3ccc(Br)cc3F)c(C(=O)NOCCO)cc21]

17. A process for the preparation of compound of formula-3, which comprises:
a) mesylation of compound of formula-11 to provide compound of formula-10; 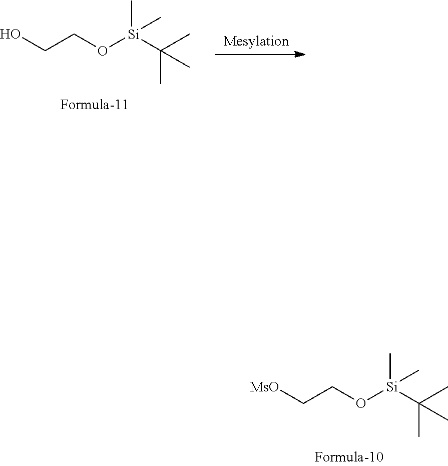
b) reacting the compound of formula-10 with N-hydroxyphthalimide to provide compound of formula-9; and 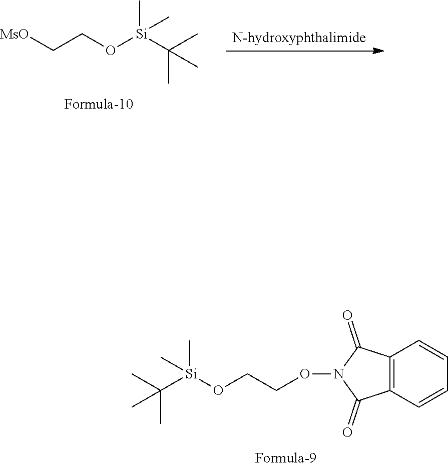 [SMILES: CC(C)(C)[Si](C)(C)OCCOS(C)(=O)=O ; CC(C)(C)[Si](C)(C)OCCON1C(=O)c2ccccc2C1=O]
c) converting the compound of formula-9 to compound of formula-3.

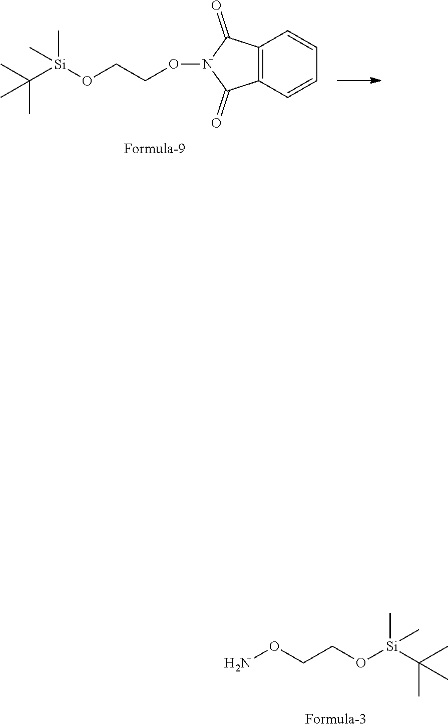 [SMILES: CC(C)(C)[Si](C)(C)OCCON1C(=O)c2ccccc2C1=O ; CC(C)(C)[Si](C)(C)OCCON]

18. A process using a compound of general formula-12 for preparing Binimetinib. 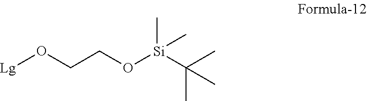
wherein, “Lg” is a leaving group such as tosyl, mesyl, acyl, benzyl and like.

19. Usage of crystalline compound of formula-2 for the preparation of Binimetinib.

20. Usage of crystalline form-M of compound of formula-2 for the preparation of Binimetinib.
